(19) United States
(12) Patent Application Publication
(10) Pub. No.: US 2015/0283324 A1
(43) Pub. Date: Oct. 8, 2015

(54) CELL DELIVERY DEVICE AND SYSTEM WITH ANTI-CLUMPING FEATURE AND METHODS FOR PELVIC TISSUE TREATMENT

(71) Applicant: AMS RESEARCH CORPORATION, Minnetonka, MN (US)

(72) Inventors: Tania M. Schroeder, Hastings, MN (US); Brian P. Watschke, Minneapolis, MN (US); Issac Marks, Minneapolis, MN (US); David J. Yonce, Edina, MN (US)

(21) Appl. No.: 14/441,505

(22) PCT Filed: Nov. 14, 2013
(86) PCT No.: PCT/US2013/070049

§ 371 Date: May 7, 2015

Related U.S. Application Data
(60) Provisional application No. 61/726,247, filed on Nov. 14, 2012.

(51) Int. Cl. A61M 5/31

(52) U.S. Cl. CPC A61M 5/3129; A61M 5/3145

(57) ABSTRACT

The invention is directed to cell delivery devices for providing a cell composition to a tissue or organ in the pelvic area for the treatment of a pelvic disorder. In some arrangements, the device has a cell delivery conduit that includes a turbulence-inducing feature that introduces sheer forces in the flow of liquid composition through the conduit, resulting in reduced cell clumping and improved single state cell delivery to the target tissue. In other arrangements, the device has a microfluidics channel which provides a similar effect for cell delivery. The resulting cell delivery can provide improved seeding of cells at the target tissue or organ and an improved therapeutic effect.

### PRIORITY

This application claims the benefit of U.S. Provisional Patent Application Ser. No. 61/726,247, filed Nov. 14, 2012, entitled CELL DELIVERY DEVICE AND SYSTEM WITH ANTI-CLUMPING FEATURE AND METHODS FOR PELVIC TISSUE TREATMENT, the disclosure of which is incorporated herein by reference.

### FIELD OF THE INVENTION

The invention relates generally cell delivery instruments and methods for treating pelvic tissue disorders.

### BACKGROUND OF THE INVENTION

Cell based therapies involve delivering cells to a tissue to treat a disorder or disease. These therapies are considered regenerative therapies aimed at restoring the function and features of healthy tissues and organs. Cell based therapies have more recently focused on the transplantation of autologous stem cells at a tissue site. To be of therapeutic benefit, transplanted stem cells should integrate into the tissue and differentiate into cells common to the tissue to restore tissue function by regeneration.

Most cells have a natural tendency to adhere to one another, which is promoted by cell adhesion molecules such as selectins, integrins, and cadherins. While cell adhesion can be important in maintaining a multicellular structure in the target tissue, it presents challenges prior to or during the transplantation event, and after cells are harvested from the body. Cells in solution have the tendency to clump together and this can cause problems in cell delivery and seeding of the cells to the target tissue.

The cell delivery devices, systems, and methods of the invention address problems and provide solutions to the problem of cell delivery and clumping in cell based therapies.

### SUMMARY OF THE INVENTION

The invention is directed to devices, systems, and methods for the treatment of pelvic tissue disorders using a cell delivery device. Cell delivery devices of the invention include those having a turbulence-inducing feature, and those having a microfluidics channel. The cell delivery devices can improve cell based therapies by preventing or disrupting the clumping of cells, thereby increasing the number of cells in the composition that are not clumped, such as a composition wherein a substantial number of cells are present in a single state. Based on this, cell compositions delivered to a patient can have improved seeding in the tissue intended to be treated, and provide a better therapeutic outcome.

Embodiments of the invention are directed to a delivery device for providing cells to a pelvic tissue. The device comprises a cell delivery conduit having a distal end configured to reach a target pelvic tissue site in a subject, an actuation member that can cause flow of a liquid composition carrying cells through the cell delivery conduit towards the distal end; and a turbulence-inducing feature. The turbulence-inducing feature is (a) positioned within a lumen of the cell delivery conduit, (b) attachable to the cell delivery conduit, or (c) formed on an inner diameter wall of the lumen of the cell delivery conduit. The turbulence-inducing features is in fluid communication with, and induces turbulence in the flow of the cell-containing liquid composition when the device is in operation.

In some embodiments, the turbulence-inducing member is formed on the inner diameter wall of the lumen of the cell delivery conduit. The member can include surface depressions or surface elevations on the inner diameter wall that are arranged in a helical configuration along all or a portion of the length of the cell delivery conduit.

In other embodiments, the turbulence-inducing member is positioned within a lumen of the cell delivery conduit and comprises a fluid deflection member affixed in the lumen having a surface that is at an angle to the central axis of the lumen. In some embodiments, the fluid deflection member has the shape of a baffle, blade, plate, or vane. In some embodiments, the fluid deflection member has a curved surface, such as a convex or concave surface. In some embodiments, the fluid deflection member comprises a propeller configuration comprising two or more blades.

Other embodiments of the invention provide a delivery system for providing cells to a pelvic tissue. The system comprises a first portion comprising a cell delivery conduit having a distal end configured to reach a target pelvic tissue site in a subject, an actuation member that can cause flow of a liquid composition carrying cells through the cell delivery conduit towards the distal end; and a second portion comprising a turbulence-inducing feature (a) positioned within a lumen of the cell delivery conduit, (b) attachable to the cell delivery conduit, or (c) formed on an inner diameter wall of the lumen of the cell delivery conduit.

In other embodiments, the invention provides another delivery device for providing cells to a pelvic tissue. The device comprises a cell solution holding chamber, a microfluidics channel in fluid communication with the cell solution holding chamber which comprises proximal and distal ends and a non-linear path between the ends, and an actuation member that can cause flow of a liquid composition carrying cells from the cell solution holding chamber and directly or indirectly into the microfluidics channel.

In other embodiments, the invention provides a method for treating a pelvic tissue disorder. The method comprises a step of delivering a composition comprising cells to a pelvic floor tissue using any device or system described herein.

In some modes of treatment, the pelvic tissue disorder treated is kidney disease. In some modes of treatment the composition comprises adipose-derived stem cells.

### BRIEF DESCRIPTION OF THE DRAWINGS

FIG. 1 is an illustration of a portion of a cell delivery conduit of a cell delivery device showing an inner jacket made of helically-wound strips.

FIG. 2 is an illustration of the distal end of a cell delivery conduit of a cell delivery device showing an inner jacket made of helically-wound strips.

FIG. 3 is an illustration of a portion of a cell delivery conduit of a cell delivery device showing a propeller-type turbulence-inducing member.

FIG. 4 is an illustration of a portion of a cell delivery conduit of a cell delivery device showing a propeller-type turbulence-inducing member and a proximally-positioned filter.

FIG. 5 is an illustration of a portion of a cell delivery conduit of a cell delivery device showing baffle-type turbulence-inducing members arranged in series.

FIG. 6 is another illustration of a portion of a cell delivery conduit of a cell delivery device showing baffle-type turbulence-inducing members arranged in series.

FIG. 7 is another illustration of a portion of a cell delivery conduit of a cell delivery device showing baffle-type turbulence-inducing members arranged in series.

FIG. 8a is an illustration of a cell delivery device having a microfluidics channel and cell storage compartment, with the microfluidics channel shown in greater detail in FIG. 8b.

FIG. 9 is an illustration of the distal end of a cell delivery conduit of a cell delivery device showing an inner jacket made of helically-wound strips.

FIG. 10 is an illustration of the distal end of a cell delivery conduit of a cell delivery device showing an inner jacket made of helically-wound strips.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Some embodiments of the invention include those directed to devices, systems, and methods for the treatment of a pelvic tissue disorder using a cell delivery device having a turbulence-inducing feature that induces turbulence in the flow of a liquid composition that includes cells. The turbulence is able to prevent cell clumping, break up clumped cells, or both, during the delivery process. In other embodiments, the device includes a microfluidics channel in which cells flow through. The microfluidics channel has a non-linear path between its proximal and distal ends through which cells flow through and which keeps the cells in an unclumped state due to the small diameter of its channel and its non-linear path.

The devices, systems, and methods of the invention can improve cell based therapies for pelvic tissue disorders by providing a composition where a greater percentage of the cells in the composition are not clumped as the composition exits the delivery end of the device. For example, more of the cells in the composition can exit the delivery device in a single cell state. This can improve seeding of the delivered cells in the tissue intended to be treated, and accordingly lead to a better therapeutic outcome.

The cell delivery device with a turbulence-inducing feature of a microfluidics channel can be a part of a system that optionally includes other components such as one or more components for obtaining and preparing a therapeutic cell composition. In some cases the therapeutic cell composition is derived from adipose tissue and the system can therefore include components for removal of adipose tissue, the enrichment of adipose derived stem cells, and/or the mixing of adipose stem cells with a cellular matrix component. Other system components which can optionally be incorporated in the system or used in optional steps of the method for treating pelvic tissue include anesthetics and antibiotics; surgical instruments such as scalpels, forceps, needles, and sutures; and bandages and tapes. The optional components can be used to numb, prevent infection, and/or repair tissue in the patient.

The cell delivery devices generally include a distal end and a proximal end. The “distal end” refers to a portion of the device from which the cell composition exits the device. In some embodiments the distal end is the end of a catheter-type of conduit, and in other embodiments the distal end can be the tip of a syringe-type of device. The distal end of the device is at the end of a distal portion of the device. In some cases, such as where the delivery conduit is of a catheter-type conduit, the distal portion can be configured to be placed and moved within the body. For example, the distal portion can be configured to move through a body lumen such as an artery or vein, or a part of the urogenital tract, such as the urethra or ureter. The distal end may also include optional functional features that operate on tissue during use, such as a frictional tissue holding tip, or a light.

In cases where the delivery conduit is a catheter-type conduit, the size of the conduit can be chosen based on factors such as the portion of the body in which the conduit is intended to travel (e.g., a body lumen such as vasculature or lumens of the urogenital system). In some cases the delivery conduit has an outer diameter (OD) in the range of about 1.8 mm to about 4.7 mm (about 4 French (Fr) to about 12 Fr), or more specifically in the range of about 1.8 mm to about 3.1 mm (about 4 Fr to about 7 Fr). Exemplary inner diameters (ID) of the delivery conduit are in the range of about 1.5 mm to about 4.1 mm, or more specifically in the range of about 1.5 mm to about 2.5 mm.

The conduit can have an external and an internal shape, for example, as viewed in a cross section of the conduit. External an internal shapes of the conduit can be the same (e.g., both are circular), or different. Other shapes include oval and polygonal, for example, hexagonal, octagonal, etc.

The “proximal end” (i.e., the end that is more towards the operator) of a cell delivery device can include an actuation mechanism that causes the flow of a cell composition though the cell delivery conduit or microfluidics channel and out the distal end of the device. The proximal end can be configured to remain external to the body. The actuation mechanism can be a mechanical feature such as the plunger of a syringe that can be manually operated to provide pressure within the delivery device and movement of a cell composition through the delivery conduit. The actuation mechanism can be controlled by a trigger or a valve, which can be manually or electronically operable, or both. Alternatively, the actuation mechanism can be associated with a pump mechanism, such as one that is electrically controlled. The proximal end can also include a reservoir for holding the cell composition prior to it being moved though and out of the device for patient treatment.

The delivery conduit can be made of a flexible or semi-rigid material, such as a flexible or semi-rigid plastic or metal material, or combinations of such material. Plastic materials that can be used to make the delivery conduit include poly(urethanes); poly(carbonates); poly(amides); poly(sulfones); poly(ethylene terephthalate); polydimethylsiloxanes; vinyls such as poly(vinyl chloride), poly(ethylene), poly(propylene), poly(vinyl acetate), poly(vinylidene difluoride); acrylics such poly(methacrylamide), and poly(acrylamide); poly(methyl acrylate), poly(methyl methacrylate), poly(acrylic acid), poly(methacrylic acid); nylons such as poly(caprolactam), poly(hexamethylene adipamide). Metals that can be included in the delivery conduit include alloys such as stainless steel, titanium/nickel, nitinol alloys, cobalt chrome alloys, non-ferrous alloys, and platinum/iridium alloys. Combinations of plastic and metal materials can be used in the conduit.

In some embodiments, the delivery conduit can include sections having different rigidities. For example, the delivery conduit can have a section with increased rigidity that houses the turbulence-inducing feature. The section with increased rigidity can be less flexible than other sections of the conduit and offer protection for the turbulence-inducing feature. Therefore, a portion of the conduit lengthwise may be structured as “A-B-A” with “A” representing a more flexible section “B” representing a less flexbile (more rigid) section, where inside section “B” of the conduit is the turbulence-inducing feature.

The section with increased rigidity can be fabricated a variety of ways. For example, a conduit made along its length of a certain flexible material or materials can be strengthened at a section by applying or forming a strengthening material such as a more rigid plastic or metal on the outer surface of the conduit. As another example, the conduit may be fabricated by molding or extrusion with the process including adding a strengthening material at the desired section.

Various embodiments of the invention provide devices having a cell delivery conduit that includes a turbulence-inducing feature. The turbulence-inducing feature can function to create turbulence in the flow of liquid that contains cells as the liquid is moving through the delivery conduit towards the distal end. In other words, the turbulence-inducing feature causes some of the liquid to move in a direction that is at an angle, or at angles, to the central axis of the delivery conduit (i.e., the central axis running parallel to the direction of the cell delivery conduit). By creating turbulence in the flow of liquid and the resulting sheer forces associated with such turbulence, cells in the liquid are less likely to adhere to one another. Further, if there is cell-cell adherence, the turbulence increases the chances that such adherence will be disrupted. As such, the liquid composition as it is moved through the cell delivery conduit having a turbulence-inducing feature may maintain cells in a single (un-adhered) state, prevent cell-cell adherence, or both. In turn, a higher percentage of cells exit the distal end of the delivery conduit in an unclumped state as compared to a delivery conduit that does not include a turbulence-inducing feature. This can provide a better therapeutic outcome as it can promote better seeding of the cells in the target tissue.

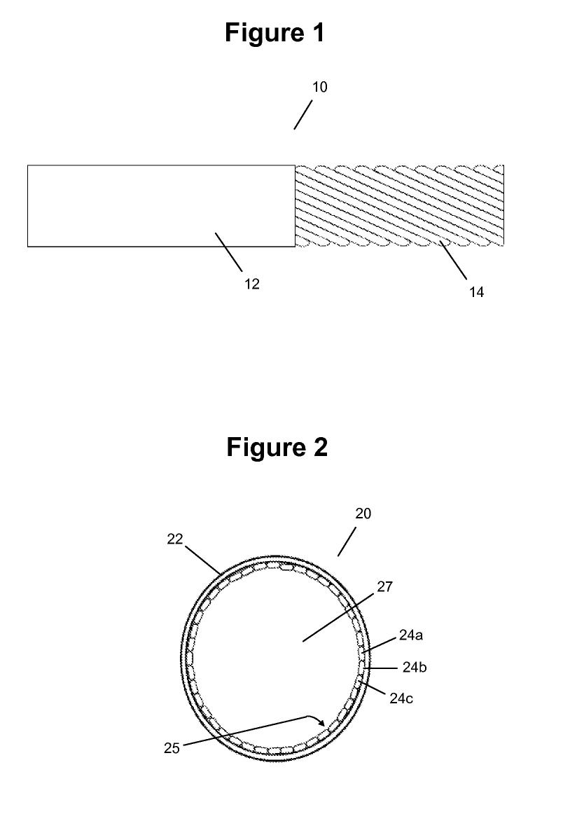

In some embodiments of the invention the turbulence-inducing feature is formed on an inner diameter wall of the lumen of the cell delivery conduit. For example, the inner wall diameter comprises surface depressions or surface elevations that are arranged in a helical configuration along all or a part of the length of the wall. Such a cell delivery conduit can be formed by a preparing a helical winding of strips or strands of material over a wire or cylinder, and then providing continuous outer sheath over the helical winding of material. The wire or cylinder on which the winding is formed is removed and the helical winding of material is formed of the inner wall of the delivery conduit, and the continuous outer sheath represents the outer wall. For example, FIG. 1 shows a portion of a delivery conduit 10 of a cell delivery device formed from a plurality of helically wound strips 14, and a continuous outer jacket 12 that covers the helically wound strips. The helical winding of strips can, in some cases, be described with regards to the angle of winding relative to the central axis of the delivery conduit. For example, in some cases strips of the winding are at an angle less than about 60° relative to the central axis, less than about 45° relative to the central axis, or less than about 30° relative to the central axis.

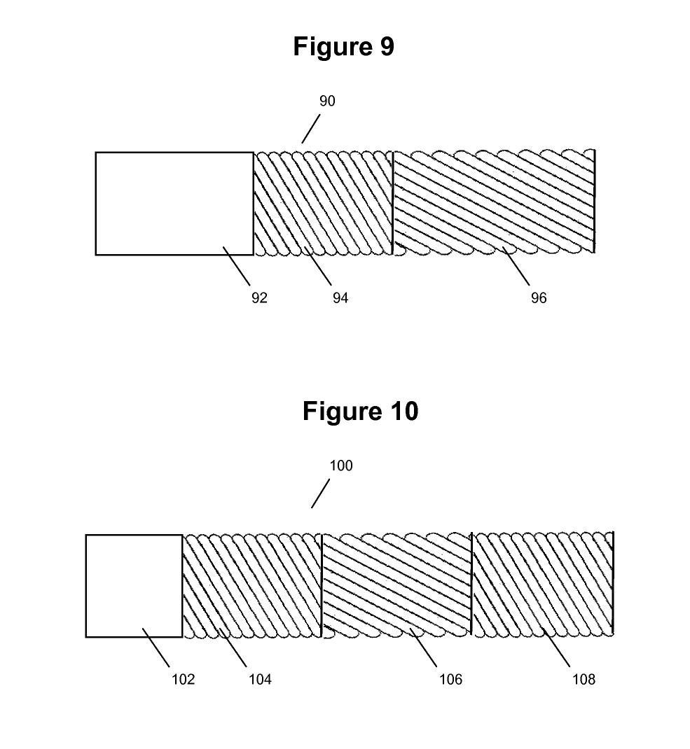

In some embodiments, the winding of the strips can change along the length of the conduit. For example, the winding can change in a proximal to distal direction causing one or more changes in the angle of the strips relative to the central axis. As a result, there can be a section of the conduit having a tighter winding (greater angle) followed by a region of looser winding (smaller angle). Along the length of the delivery conduit the winding can alternate from tight to loose, and optionally back to tight. The change in winding can be gradual or abrupt. For example, during manufacture the different winding can be started at different points along the length of the conduit. Variation in the winding of the strips can induce more turbulence and cell separation by changing the direction of deflection of the fluid path though the conduit. For example, FIG. 9 shows a portion of a delivery conduit 90 of a cell delivery device formed from a first section having a plurality of helically wound strips 94 with a tight winding, a second section having a plurality of helically wound strips 96 having a looser winding, and a continuous outer jacket 92 that covers the helically wound strips. The angle of the helically wound strips relative to the central axis in the first section is greater than the second section. FIG. 10 shows a portion of a delivery conduit 100 with three sections of helically wound strips (104, 106, and 108) having tight, loose, and then tight windings, respectively. The differences in the angles helically wound strips relative to the central axis between different sections can be greater than about 5°, greater than about 10° greater than about 15°, or greater than about 25°, such as in the range of about 5° to about 60°, or in the range of about 10° to about 45°.

FIG. 2 illustrates the delivery conduit 20 as seen from the distal end. The delivery conduit 20 has an outer wall 22, and a plurality of helically wound strips (e.g., 24a, 24b, 24c, etc.) forming the inner wall. Between the strips, along the length of the helical winding, are grooves 25, which may also be referred to as troughs. The grooves can be of any size or shape so as to provide an inner wall that can induce a turbulent flow when fluid is moved down the delivery conduit. The troughs or grooves can induce a rotating flow of the liquid along the inner diameter wall as the liquid cell composition is moved down the length of the cell delivery conduit. The spin induces a turbulent flow in a vortex manner which can prevent cell-cell attachment, can break up attached clumps of cells, or both, caused by the sheer forces within the liquid flow.

In other embodiments of the invention, the turbulence-inducing feature is positioned within a lumen of the cell delivery conduit. As a general matter, the turbulence-inducing feature can deflect the flow of the fluid carrying the cells as it travels down the delivery conduit and can induce turbulence in the liquid. The turbulence-inducing feature can include one or more surfaces that are at an angle to the central axis of the delivery conduit. The surfaces of the turbulence-inducing member can be flat or curved, or if there are multiple surfaces a combination of flat and curved surfaces can be used. The member can be affixed in the lumen so that that the flow of liquid does not force the member out of the delivery conduit. The member however, can, in some embodiments, have parts that move in position within the lumen. For example, the turbulence-inducing member can have parts that rotate in place, such as with propeller motion, or that flap, such as with rudder motion, when fluid travels down the delivery conduit and passes over the angled surface of the turbulence-inducing member.

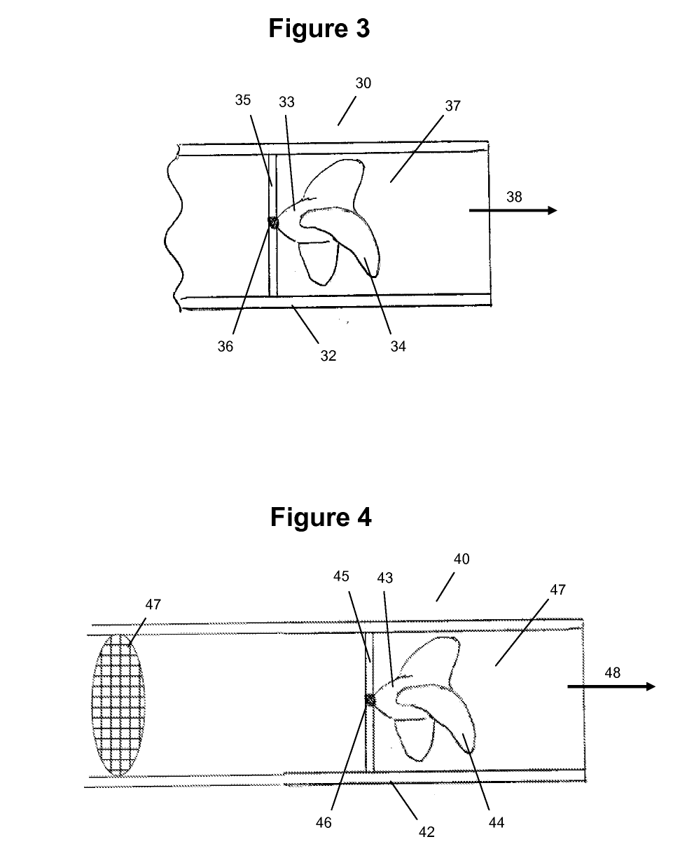

In some embodiments the turbulence-inducing member includes a propeller configuration with the member comprising two or more blades, such as two, three, four, or five blades. As an example, FIG. 3 shows a portion of a delivery conduit 30 of a cell delivery device having a conduit wall 32 and a conduit lumen 37, and a propeller-shaped turbulence-inducing member 33 affixed in the lumen. The propeller-shaped turbulence-inducing member 33 can be affixed in the conduit to a strut 35 that is attached to and that traverses the conduit wall 32. The tip 36 of the propeller-shaped turbulence-inducing member 33 can be attached to the strut 35 in a manner that allows its free rotation when fluid is moved down the delivery conduit in direction 38. The propeller-shaped turbulence-inducing member can be affixed in the delivery conduit at a desired location, for example near the distal end of the conduit, near the proximal end of the conduit, or near the central portion of the conduit. In some embodiments two or more propeller-shaped turbulence-inducing members can be affixed in the delivery conduit at desired locations.

In embodiments, the turbulence-inducing member including propeller blades is made of a rigid plastic material such as polysulfone, polyetheretherketone, polyphenylene, polyurethane, or an alloys such as stainless steel, titanium/nickel, nitinol alloy, cobalt chrome alloy, non-ferrous alloy, or platinum/iridium alloy, such as described herein.

In some embodiments the delivery conduit comprises a filter or mesh and a turbulence-inducing member. The filter or mesh can be placed at a desired location in the conduit in relation to the turbulence-inducing member. In some arrangements the filter or mesh is proximal to the turbulence-inducing member, such as shown in FIG. 4. FIG. 4 shows a propeller-shaped turbulence-inducing member 43 (conduit wall 42, conduit lumen 47, strut 45, tip 46 are also shown), but other turbulence-inducing member designs could be used in combination with a filter or mesh. In FIG. 4, the filter or mesh 47 can be positioned proximal (“upstream”) of the propeller-shaped turbulence-inducing member 43 and can function to filter out larger clumps of cells from the cell composition before the composition is passed by the propeller-shaped turbulence-inducing member 43. For example, larger clumps of cells can be removed that would not otherwise be able to be sufficiently disrupted by the sheer forces in the lumen 47 at the distal end of the delivery conduit 40. The filter or mesh can be chosen to have a pore size to allow the passage of single cells, or smaller clumps of cells that may be disaggregated when passed by the propeller-shaped turbulence-inducing member 43. Exemplary filters can have pore sizes of about 25 μm or greater, 50 μm or greater, 75 μm or greater, or 100 μm or greater, and are made from nylon, polycarbonate, ePTFE,

In some embodiments the turbulence-inducing member comprises a baffle configuration comprising one or more surfaces arranged at an angle or angles to the central axis of the delivery conduit. The baffle configuration (or baffle configurations) can in essence cause the flow of fluid carrying the cells to divide when it meets a proximal edge of the baffle and then remix further down the delivery conduit, thereby inducing turbulence in the liquid stream.

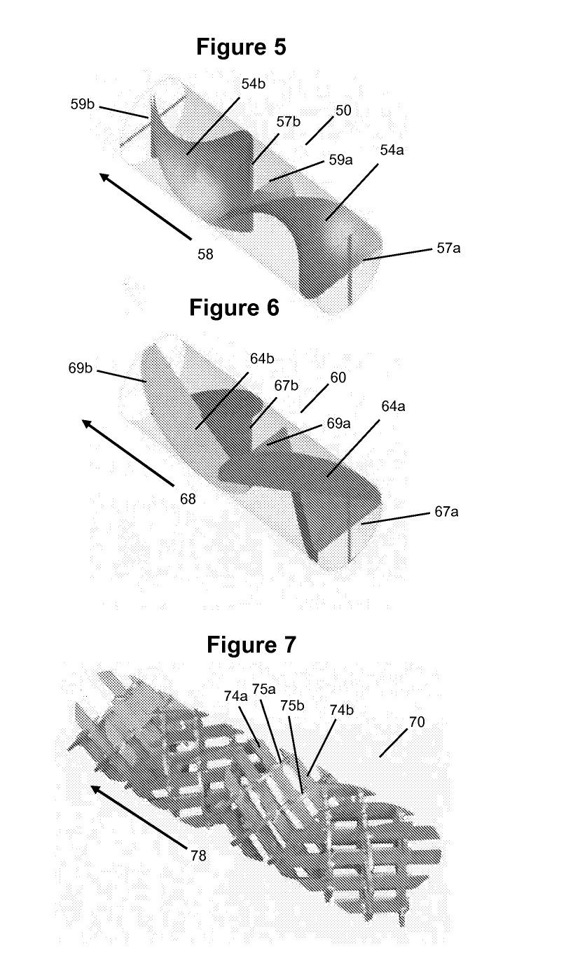

As an example, FIG. 5 shows an internal portion of a delivery conduit 50 of a cell delivery device having first and second turbulence-inducing members (54a and 54b) arranged in series and having curved surfaces. First member 54a has a half-arc shape with a proximal edge 57a that traverses the inner diameter of the lumen of the delivery conduit, a curved surface that deflects the fluid (moving in direction 58 arrow) towards the inner wall of the conduit, and a distal edge 59a, which also traverses the inner diameter of the lumen. In some arrangements distal edge 59a can be parallel to proximal edge 57a. Second member 54b can also have a half-arc shape with a proximal edge 57b, and a distal edge 59b. In some arrangements distal edge 59a of the first member 54a can be at an angle to, or perpendicular to proximal edge 57b of second member 54b.

As another example, FIG. 6 shows an internal portion of a delivery conduit 60 of a cell delivery device having first and second turbulence-inducing members (64a and 64b) arranged in series and having curved surfaces. For example, first member 64a and second member 64b have a corkscrew or helical shape with proximal and distal edges (67a, 67b, and 69a, 69b, respectively) that traverse the inner diameter of the lumen of the delivery conduit. In some arrangements distal edge 69a of the first member 64a can be at an angle to, such as perpendicular to proximal edge 67b of second member 64b.

As yet another example, the delivery conduit comprises one or more geometric grids configured to fit in the lumen of the delivery conduit. With reference to FIG. 7, a geometric grid 70 comprises a first set of elongate slats comprising two or more elongate slats (74a, 74b) arranged in the same plane or a parallel plane, and connected to and separated by a second set of elongate slats comprising two or more elongate slats (75a, 75b) which are arranged at an angle (e.g., such as perpendicular) to the first set of elongate slats. In this arrangement, the configuration of slats provides multiple edges which deflect the flow of fluid carrying the cells, causing turbulence, and promoting the disruption of cells clumps and a higher percentage of single cells in the delivery composition that exit the delivery conduit.

The turbulence-inducing member can be sized to fit within the inner diameter of the delivery conduit. In some cases, the turbulence-inducing member can be described in terms of one or more of its dimensions, such as length (e.g., as measured along the central axis of the delivery conduit) and width (e.g., as measured in a line perpendicular to the central axis of the delivery conduit). For example, the turbulence-inducing member can have a dimension (such as a width) that is equal to or less than the inner diameter of the delivery conduit. In some embodiments, the turbulence-inducing member can have a width that is about 2.5 mm or less, about 2.2 mm or less, about 1.9 mm or less, or about 1.5 mm or less. In some embodiments the turbulence-inducing member has a length that is greater than its width.

The turbulence-inducing feature can be made from any biocompatible material, such as biocompatible metals or plastics. The term “biocompatible” means there is not an adverse impact on the cells in the composition. In some embodiments the turbulence-inducing feature is made partially or entirely or a non-adherent material, which can generally prevent cells from adhering to the surface of the member. For example, a non-adherent material can be a hydrophobic plastic material such as polytetrofluoroethylene (PTFE).

The turbulence-inducing feature can have a surface that is modified to prevent cell adherence, or modified to increase cell repulsion. The option of modifying the surface turbulence-inducing feature can be made based on the type of material or materials used to fabricate the turbulence-inducing feature. One type of modification is the formation of an inert hydrophobic surface on the turbulence-inducing feature.

Hydrophobic surfaces can be formed on a turbulence-inducing feature using hydrocarbon and fluorocarbons materials. Hydrocarbon and fluorocarbon materials can be plasma polymerized to form thin highly hydrophobic films on the surface turbulence-inducing feature. A process for forming a thin hydrophobic film on the surface can include heating a fluorocarbon monomer so that it pyrolyzes and produces reactive species in the vicinity of the structure surface, where the monomer gets deposited on the surface and forms a thin film. Exemplary processes for forming a thin film are described in U.S. Pat. No. 5,888,591.

Fluorocarbon monomers that can be used to form a thin film include, but are not limited to C₂F₄, C₃F₈, CF₃H, CF₂H₂, difluorohalomethanes such as CF₂Br₂, CF₂HBr, CF₂Cl₂, and CF₂FCl; and difluorocyclopropanes such as C₃F6, C₃F₄H₂, and C₃F₂Cl₄.

Another material that can be formed on the surface of a turbulence-inducing feature is poly(ethylene oxide) (PEO). PEO can reduce the absorption of proteins and adhesion of cells to surfaces. PEO can be attached to a surface of a turbulence-inducing feature by absorption to a hydrophobic surface, or by covalent coupling of modified PEO molecules (e.g., see Desai, N. P., and Hubbel, J. A. (1990) ACS Polym. Mater. Sci. Eng. 62:731) or grafting to a polymeric surface via a backbone polymer (e.g., see Nagaoka, S. et al. (1985) Polymers as Biomaterials, pp. 361, Plenum Press, New York)

Other embodiments of the invention provide a cell delivery device that includes a microfluidics channel. The microfluidics channel has a non-linear path, which include abrupt path direction changes, between its proximal and distal ends through which cells flow and which keeps the cells in an unclumped state due to the small diameter of the channel and the deviations in direction along its path. The microfluids channel has a diameter greater than the average diameter of a mammalian cell (e.g., greater than about 10 μm), or can have a diameter greater than about 25 μm, greater than about 50 μm, greater than about 75 μm, or greater than about 100 μm. The microfluids channel can have diameter less than about 1 mm, less than about 750 μm, or less than about 500 μm. Exemplary diameters for the microfluidics channel are in the range of about 10 μm to about 1 mm, about 25 μm to about 750 μm, or about 50 μm to about 500 μm.

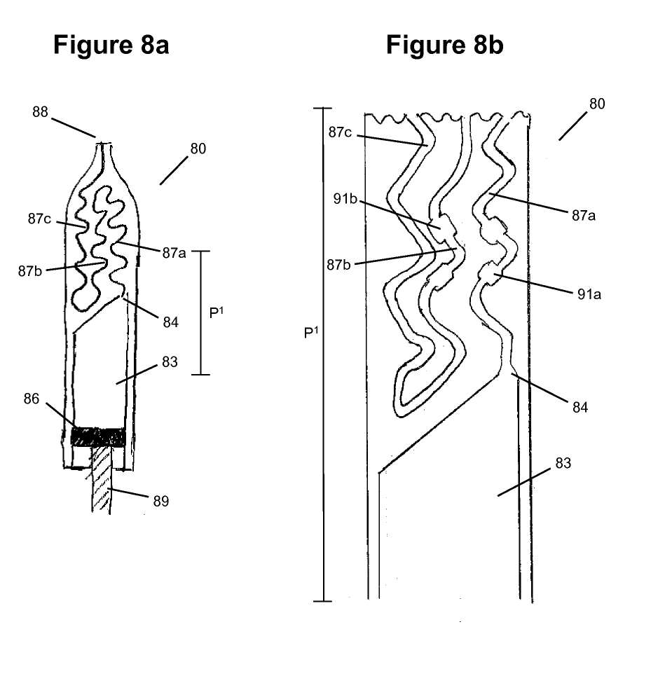

An exemplary cell delivery device with a microfluidics channel is shown in FIG. 8a. The cell delivery device 80 can have a cell chamber 83 for holding a liquid composition of cells, plunger/stopper members (89, 86) at the proximal end of the device and movable within the cell chamber 83 to pressurize the liquid composition to cause its movement into the microfluidic channel 87 (e.g., represented by portions 87a-c of the microfluidics channel), starting at entry port 84 (proximal end of the microfluidics channel), and a distal end of the device having an exit aperture 88 (distal end of the microfluidics channel) from which the cell composition is dispensed. In some embodiments the cell delivery device 80 can have a size of a standard syringe, and the cell chamber 83 can be sized for holding a volume of cell composition for treating a target tissue or organ. For example the cell chamber 83 can hold a volume in the range of about 500 μL to about 100 mL, or about 1 mL to about 50 mL.

The microfluidics path 87 can have multiple deviations in various directions, such as shown in FIG. 8a, and in greater detail in FIG. 8b. The microfluidics path 87 can move, overall, in a proximal to distal direction in the cell delivery device 80, or can move in both proximal to distal, and distal to proximal directions in the cell delivery device 80. For example, portion 87a moves in generally a proximal to distal direction (with back and forth changes in direction in this portion); portion 87b moves in generally a distal to proximal direction (with back and forth changes in direction in this portion); and portion 87c moves fluid in generally a proximal to distal direction (with back and forth changes in direction in this portion), exiting at the distal end of the device, aperture 84.

In some embodiments the microfluidics channel can include portions where the diameter of the channel increases. For example, referring to FIG. 8b the microfluidics channel can include one or more microreservoirs (91a, 91b) located at desired location(s) along the microfluidics path. By including a microreservoir, there is a change in channel diameter from narrow to wider and then back to narrow, which can lead to changes in velocity of the cell composition travelling through the microfluidics channel which can also promote the breaking up of cell clumps, or prevent cells from adhering to one another.

The cell delivery devices of the invention can have a distal end from which the composition containing cells is dispensed, such as to a desired tissue in a patient. In some cases the cell delivery devices dispense the cell composition from a needle located on the distal end of the device. In some arrangements, the distal end of the device can include multiple needles, multiple apertures in a single needle, or multiple apertures among multiple needles. Embodiments that include multiple apertures or multiple needles can include an extended, expanded, or extendable chain, string, array, or sequence (e.g., “daisy chain”). Apertures may be located at an extension mechanism (“aperture extension”) such as extendable or fanning needles.

The fluid composition containing cells can be dispensed from the distal end of the cell delivery device to a tissue or organ using a desired velocity, pressure, and volume sufficient to provide a desired number of cells to the treatment site. The duration of dispensing the liquid composition can be performed as desired. In some methods of dispensing, the duration of dispensing is controlled by one or more feature(s) of the cell delivery device, such as a solenoid or valve, in order to meter the flow of the composition through the cell delivery conduit of microfluidics path, and out the distal end of the device. Delivery of the cell composition can be performed in a single treatment period, or over multiple treatment periods.

In some modes of practice, the dispensed cells can seed into the target tissue and exert a therapeutic effect. For example, the seeded cells may in some cases regenerate damaged tissue, or in other cases, promote re-vascularization of tissue. In some modes of practice, the cell delivery device is used for the treatment of kidney disease, such as acute or chronic kidney diseases (such as described in Mollura, D. J., et al. (2003) Stem-cell therapy for renal diseases. Am J Kidney Dis. 42:891-905). For example, systemically introduced stem cells can engraft in sites of renal disease and injury to show donor phenotypes. Stem cells can differentiate into cells similar to glomeruli, mesangium, and tubules in the kidneys.

The device and methods of the invention can be used to treat kidney diseases such as proteinuria (albuminuria), diabetic nephropathy, polycystic kidney disease (PKD), chronic kidney disease (CKD), and autoimmune glomerulonephritis. Proteinuria (albuminuria), which is a condition in which urine contains an abnormal amount of protein, and which is thought to result from damaged glomeruli of the kidney. As another example that can be treated, diabetic nephropathy is a progressive disease where the capillaries in the kidney glomeruli undergo angiopathy, and caused by diabetes mellitus. As another example, polycystic kidney disease (PKD) is a cystic genetic disorder of the kidneys. Chronic kidney disease (CKD) is also characterized by accumulation of extracellular matrix. Autoimmune glomerulonephritis is associated with a significant immune response with glomerular crescentic formation and fibrosis in the kidney.

Stem cells can exhibit self-renewal and are able to differentiate into specialized cell types. In one mode of practice, adipose derived cells (ADCs) are removed from adipose tissue and introduced to the treatment region using the cell delivery device. Adipose (i.e., fat) tissue includes or yields a high number of desirable cell types, including stem cells. Systems and methods of the invention can optionally include devices, tools, and methods for the preparation of a composition containing a cell population derived from adipose tissue. To obtain an adipose tissue sample, a lipectomy surgical procedure can be performed. Adipose tissue obtained by lipectomy can be processed and then the cell preparation obtained can be reintroduced into the tissue of the same patient, thereby providing an autologous source of cells.

The adipose tissue can come from anywhere in the body. In one embodiment, the adipose tissue is obtained from the abdominal area of the patient. Other common areas may include the thigh and back area of the patient. To provide an adequate amount of cells, adipose tissue in an amount in the range of about 60 cc to about 120 cc is obtained from the patient. Optionally, if desired, a portion of the adipose tissue is set aside for preparing a “cell matrix” which can be remixed with an enriched population of cells from the adipose tissue.

In some modes of practice, adipose tissue is processed to separate the adipose-derived stem cells from the other material including other cellular and non-cellular material in the adipose tissue. Preparation methods can include steps of washing the tissue, treating the tissue with collagenase or trypsin, or optionally with mechanical agitation. Liposomes, which are generally aggregated, can be separated from free stromal cells which include the stem cells and other cells such as red blood cells endothelial cells, and fibroblast cells, by centrifugation. Erythrocytes can be lysed from the suspended pellet and the remaining cells can be filtered or centrifuged. Optionally, cells may be separated by cell sorting or separated immunohistochemically. Methods for the preparation of adipose-derived stem cells are described in commonly-assigned application number WO 2009/120879.

In other modes of practice, the adipose tissue is processed to remove partially or substantially non-cellular components, and to form a heterogenous cell mixture. The heterogenous cell mixture can include endothelial cells, endothelial precursors and progenitors, mesenchymal stem cells, vascular smooth muscle cells, fibroblasts, pericytes, macrophages, and the like.

PCT Application PCT/US2010/041508 describes methods and apparatus for the preparation of cellular material useful for introduction to a target tissue using the cell delivery device of the invention. Cell separation equipment is also commercially available from, for example, Tissue Genesis, Inc. (Honolulu, Hi.).

In some modes of practice, stem cells can be treated with one, or a combination of different factors, to promote differentiation of cells towards a desired cell type. Stem cells can be treated with the one or more factors in vitro for a desired period of time, and then delivered to the tissue intended to be treated. For example, for the treatment of kidney disease, stem cells can be treated with nephrogenic growth factors to promote differentiation of stem cells into renal epithelial cells. Such differentiation may improve the ability of the cells to integrate into a tissue for regeneration. Exemplary factors which may promote differentiation include small lipophilic molecular ligands for receptors, and peptide and protein involved in cell activation. For example, a composition comprising retinoic acid, Activin-A, and Bmp7 can be used to induce in stem cells the expression of markers specific for the intermediate mesoderm, from which the kidneys arise (e.g., see Kim, D., and Dressler, G. R. (2005) J. Am. Soc. Nephrol., 16:3527-3534)

After a population of the adipose-derived cells (e.g., stem cells) is enriched and optionally treated with differentiation factors in vitro, the cells can be introduced into a tissue or organ of the pelvic area using the cell delivery device. Optionally, in other modes of practice, the adipose-derived cells are mixed with one or more materials that provide a “cell matrix” for the injected cells. The cell matrix can be chosen from synthetic components, natural components, or mixtures thereof, and can improve one or more of the following properties at the site of injection: cell viability, cell retention, cell differentiation, and cytokine production. Optional cell matrices include platelet rich plasma (PRP) or platelet poor plasma (PPP). PRP is blood plasma enriched with platelets. Through degranulation of the platelets, PRP can release different cytokines that can stimulate healing of soft tissue. Processes for PRP preparation include the collection of centrifugation of whole blood which separates PRP from platelet-poor plasma and red blood cells. In some cases, the adipose-derived stem cells are combined with PRP and delivered to a target tissue using the cell delivery device of the invention. PRP also includes many regenerative proteins to hasten healing. The adhesive or retention function of PRP can prevent cells from migrating or being lost through body fluid flow.

Another optional cell matrix includes platelet poor plasma (PPP). PPP is typically characterized by a very low number or platelets (<50000/uL) and a high concentration of fibrinogen. PPP can be prepared in a centrifugation process that separates it from PRP and red blood cells. PPP can provide an autologous scaffold-like material to keep injected cells local to the target tissue to improve the regenerative potential of the cells. PPP can be beneficial to tissue as well. The PPP can include a porous gelatinous material to keep cells local to the injection site and provide a therapeutic effect. PPP can allow the movement of cytokines and other signaling molecules in and out of the tissue for regenerative mechanisms local to the injection site.

In some modes of practice, the optional cell matrix is prepared from a portion of the adipose tissue obtained from the patient. To prepare the cell matrix, the adipose tissue can be disaggregated by mechanical force, such as by cutting, chopping, or mincing the adipose tissue. Generally, for this cell matrix preparation, collagenase or trypsin (enzymatic) digestion is not performed to maintain the scaffolding features of the adipose tissue. The adipose particles generated using such a process are sized for use in cell compositions for tissue or organ treatment. Grinding and filtering parameters can also be employed depending on the particular treatment site needs.

In some preparations, the cells are mixed with the disaggregated adipose tissue at a weight ratio in the range of about 1:1 to about 1:4. Methods for the preparation of an adipose tissue-derived scaffolding for cells are described in commonly assigned International Application PCT/US2009/038426 (WO2009/120879).

In some modes of therapy, the cell matrix component is delivered to the tissue prior to delivery of the cells, after delivery of the cells, or in a manner that is not strictly synchronous with cell delivery. For example, an amount of cell matrix component, without cells, can be delivered to the tissue first, followed by a mixture of the cells and the cell matrix component.

The cell-containing composition can optionally include biologics or drugs which can enhance the effectiveness of the cells following delivery of the composition to a target tissue, or that can further improve the condition of the tissue. Optionally, the cell-containing composition can include excipients, additives, or auxiliary substances such as an antioxidants, antiseptics, isotonic agents, and buffering agents.

In some aspects of the invention, the cell delivery device with turbulence-inducing feature of microfluidics channel can be optionally be used in conjunction with a multi-chamber cell mixing system, such as described in commonly assigned U.S. Publication No. 2012/0156178. For example, multi-chamber cell mixing system can be attached to the cell delivery conduit having a turbulence-inducing feature, or a microfluidics channel as described herein. For example, a multi-chamber cell mixing system can include various components and elements to facilitate mixing, digesting, filtering and cellular mixtures, e.g., cells and autologous adipose tissue or scaffolding material.

In some arrangements, a cell mixing system and delivery system can include a first syringe chamber, a second syringe chamber, and mixing element, attached to the cell delivery conduit having a turbulence-inducing feature, or a microfluidics channel as described herein needle. The first syringe chamber can include an interior portion or lumen defined therethrough and can further include an inlet port or opening, and the second syringe chamber can include a grinder or digestion element (e.g., grinder, mincer or chopper device), as well as a filter or mesh element. The grinder element can include spinning blades or members, and can be driven mechanically, manually or electrically. The filter element can be a static or dynamic device. The second syringe chamber can further include an inlet port or opening. The first syringe chamber is generally adapted to receive and advance various cells, while the second syringe chamber is adapted to receive and advance scaffolding tissues, such as adipose.

In another arrangement, a mixing system includes a first syringe chamber and a second syringe chamber which are arranged side-by-side, and lead into a common conduit prior to entering a mixing element. The system can also includes a grinder or digestion element, a filter or mesh element, a cell inlet port, and an adipose tissue inlet port. The mixing system can be attached to a cell delivery conduit with turbulence-inducing feature, or a microfluidics channel.

In some modes of practice, a portion of the adipose tissue that is obtained from the patient can be washed and processed via the second chamber, while the first chamber receives the heterogeneous or enriched cell (e.g., adipose derived stem cell) population that has been processed as described herein. Adipose tissue or particles within the second syringe chamber can be reduced in size at the grinder element, and then passed through the filter or mesh element. As such, adipose tissue of varying sizes and shapes can be reduced to a desirable and predefined dimension before passing through for mixing with the cells of the first syringe chamber at the mixing element.

The mixing element can be in fluid and operative communication with the first syringe chamber, the second syringe chamber, and the cell delivery conduit having a turbulence-inducing feature, or a microfluidics channel. The mixing element can ensure the cellular mixture does not separate prior to injection into the treatment site. Various known components, structures and techniques can be used to mix and retain the cellular mixture of adipose and cells received from the chambers into the mixing element prior to injection into the target tissue through the cell delivery conduit or microfluidics channel.

Devices, methods, and compositions prepared therefrom, including those disclosed in U.S. Patent Publication Nos. 2005/0177100, 2006/0100590, 2007/0224173, 2008/0014181, 2008/0287879 and 2009/0018496; U.S. Pat. No. 7,101,354; and PCT International Patent Publication No. WO2008/091251 can optionally be used in conjunction with the cell delivery device and methods of the current invention, and their disclosures are incorporated herein by reference in their entirety.

1. A cell delivery device for providing cells to a pelvic tissue, the device comprising:
a cell delivery conduit having a distal end configured to reach a target pelvic tissue site in a subject;
an actuation member that can cause flow of a liquid composition carrying cells through the cell delivery conduit towards the distal end; and
a turbulence-inducing feature (a) positioned within a lumen of the cell delivery conduit, (b) attachable to the cell delivery conduit, or (c) formed on an inner diameter wall of the lumen of the cell delivery conduit, that is in fluid communication with, and that induces turbulence in the flow of, liquid composition when the device is in operation.

2. The cell delivery device of claim 1 wherein the turbulence-inducing member is formed on an inner diameter wall of the lumen of the cell delivery conduit and comprises surface depressions or surface elevations on the inner diameter wall that are arranged in a helical configuration along all or a part of the length of the cell delivery conduit.

3. The cell delivery device of claim 2 wherein the depressions are in the form of grooves, troughs, or channels, or the elevations are in the form of ridges or crests, on the inner diameter wall.

4. The cell delivery device of claim 2 wherein the cell delivery conduit comprises a continuous polymeric outer jacket formed over a helical winding of strips, cords, or strands of material, the helical winding forming the inner diameter wall.

5. The cell delivery device of claim 1 wherein the turbulence-inducing member is positioned within a lumen of the cell delivery conduit having a central axis, the turbulence-inducing member comprising a fluid deflection member affixed in the lumen having a surface that is at an angle to the central axis.

6. The cell delivery device of claim 5 wherein the fluid deflection member is selected from the group consisting of a baffle, blade, plate, and vane.

7. The cell delivery device of claim 5 wherein the fluid deflection member has a curved surface (e.g., convex or concave).

8. The cell delivery device of claim 5 wherein the fluid deflection member comprises a propeller configuration comprising two or more blades.

9. The cell delivery device of claim 5 wherein the fluid deflection member comprises two or more baffles arranged in series in the fluid delivery conduit.

10. The cell delivery device of claim 1 further comprising a filter positioned in fluid communication with the liquid composition when the device is in operation.

11. The cell delivery device of claim 10 wherein the filter is positioned proximal to the turbulence-inducing feature.

12. The cell delivery device of claim 1 wherein the fluid delivery conduit has an inner diameter in the range of 1.5 mm to 2.5 mm.

13. (canceled)

14. The cell delivery device of claim 1 wherein the actuation member comprises a plunger.

15. The cell delivery device of claim 1 wherein the actuation member comprises an electric pump and a solenoid valve.

16. A delivery system for providing cells to a pelvic tissue, the system comprising:
a first portion comprising a cell delivery conduit having a distal end configured to reach a target pelvic tissue site in a subject; and
an actuation member that can cause flow of a liquid composition carrying cells through the cell delivery conduit towards the distal end; and
a second portion comprising a turbulence-inducing feature (a) positioned within a lumen of the cell delivery conduit, (b) attachable to the cell delivery conduit, or (c) formed on an inner diameter wall of the lumen of the cell delivery conduit, that is in fluid communication with, and that induces turbulence in the flow of, liquid composition when the device is in operation.

17. A delivery device for providing cells to a pelvic tissue, the device comprising:
a cell solution holding chamber;
a microfluidics channel in fluid communication with the cell solution holding chamber, the microfluidics channel comprising proximal and distal ends, wherein the channel comprises non-linear path between the proximal and distal ends; and
an actuation member that can cause flow of a liquid composition carrying cells from the cell solution holding chamber and directly or indirectly into the microfluidics channel.

18. The delivery device of claim 17 wherein the microfluidics channel comprises a diameter in the range of 25 μm to about 750 μm.

19. The delivery device of claim 17 wherein the proximal end of the microfluidics channel is connected directly to the cell solution holding chamber.

20. The delivery device of claim 17 wherein the microfluidics channel comprises one or more portions having an increase in diameter in the channel path.

21. A method for treating a pelvic tissue disorder comprising a step of delivering a composition comprising cells to a pelvic floor tissue using the delivery device of claim 1.

22-29. (canceled)
